(19) United States
(12) Patent Application Publication
(10) Pub. No.: US 2021/0381979 A1
(43) Pub. Date: Dec. 9, 2021

(54) APPARATUS AND METHOD FOR MULTIPLEXED ROTATING IMAGING BIOASSAYS

(71) Applicant: VERSITECH LIMITED, Hong Kong (CN)

(72) Inventors: Kin Man, Kevin TSIA, Hong Kong (CN); Hin Long TANG, Hong Kong (CN); Kam Seng LAU, Kowloon, Hong Kong (CN)

(73) Assignee: VERSITECH LIMITED, Hong Kong (CN)

(21) Appl. No.: 16/331,420

(22) PCT Filed: Sep. 7, 2017
(86) PCT No.: PCT/CN2017/100841

§ 371 Date: Mar. 7, 2019

Related U.S. Application Data
(60) Provisional application No. 62/384,564, filed on Sep. 7, 2016.

(51) Int. Cl. G01N 21/64; B01L 3/00; C12N 5/00; G01N 33/543

(52) U.S. Cl. CPC G01N 21/6428; B01L 3/5085; G01N 21/6456; G01N 33/54306; C12N 5/0068

(57) ABSTRACT

Systems and method for versatile multiplexed spinning/rotating bioassays are provided. This bioassay platform can take the advantage of the high-speed spinning motion, which naturally provides on-the-fly cellular imaging at the rate that cannot be reached by the conventional cameras or laser-scanning techniques, but ultrafast imaging modalities. More importantly, the functionalized solid substrates derived from the disk substrate can be compatible with adherent cell culture as well as biochemically-specific cell-capture, which can now be assayed with ultrafast imaging modalities at an ultra-high-speed line-scan rate of >10 MHz. Large-format spinning high-throughput imaging assay could thus be a potent tool for scaling both the assay throughput as well as content/multiplexity as demanded in many applications, e.g. drug discovery, and rare cancer cell screening.

### FIELD OF THE INVENTION

The present invention relates to apparatus and methods for expediting bioassays and increasing the amount of information collected.

### BACKGROUND OF THE INVENTION

Current bioassay technologies can be generally classified into three categories, in terms of the types of target specimens: (1) biomolecular (affinity) assay (e.g. DNA/protein microarray, ELISA/EIA), (2) cell-based assay (e.g. flow cytometry, imaging cytometry) and (3) tissue-based assay (e.g. tissue micro-array (TMA) and whole slide imaging (WSI)). Typical assay strategies involve: (a) suspension assay and (b) solid-substrate assay. These technologies typically have a fundamental trade-off between measurement throughput (i.e. efficiency) and measurement content (i.e. precision/accuracy). An attempt to reconcile this trade-off can be exemplified by the emerging interests in adding imaging capability in flow cytometry, the gold standard for cellular assays.

Although accessing additional spatial information about individual cells, these imaging flow cytometers only achieve an imaging throughput of ˜1,000 cells/sec, orders-of-magnitude slower than the non-imaging flow cytometers (100,000 cells/sec). In contrast to flow cytometry in which the bio-specimens are in suspension in a fluid, imaging cytometry has been another widespread approach to performing high-content measurement of isolated single cells or the bulk tissue matrix, where the specimens are mostly attached on a solid substrate. Imaging cytometry is capable of delivering high-content quantitative high-resolution image analysis in real-time (such as time-lapse measurement for cell-cycle studies, drug screening experiments, etc.). However, the measurement throughput is limited to a handful of cells (100's-1,000's per single-shot field-of-view). Enlarging the measurement (imaging) area can be achieved by mechanically scanning across the entire specimen. This is the common strategy adopted in not only imaging cytometry, but also WSI and TMA, which are emerging technologies for digital pathology as well as drug screening. High-throughput screening in the pharmaceutical industry has been extended to TMAs (more than 1,000 cores from a single tissue block) which can be employed with a wide range of techniques including histochemical, immunohistochemical, and immunofluorescent staining, or in situ hybridization for either DNA or mRNA.

The WSI and TMA techniques are related to tissue-based assays. Such assays have rapidly gained in popularity in routine pathological diagnosis because they enable automated tissue section scanning and digitization—relieving the large burden of manual inspection of large numbers of tissue sections in clinical laboratories and hospitals. However, the throughput of such techniques is limited by the common raster scanning speed of the sample stage, which is also closely linked to the highest achievable frame rate of the camera technologies utilized. Typical scanning in WSI or TMA is on the order of a two-dimensional (2D) area of <10 mm² during a time of more than 1 min, in order to maintain the image spatial resolution as high as ˜1 μm. Again, this is a clear example of the throughput-versus-content trade-off that exists in the current assay technologies. The throughput is further reduced severely when the imaged tissue is in three-dimensional (3D), e.g. 3D tissue block/scaffold or the 3D tissue prepared by tissue clearing techniques, as additional axial image scan is needed.

Furthermore, a vast majority of the current bioassay technologies rely on the use of molecular-specific biomarkers/contrast agents to enhance the measurement specificity and accuracy. Examples are the immunofluorescence labels used for flow cytometry and image cytometry; as well as the use of immunohistochemical stains (e.g. hematoxylin and eosin (H&E) stain) for histopathological examination of tumors. These molecular-specific biomarkers/contrast agents have been the major workhorses in life science research as well as biomedical diagnostics. They have proven to be useful tools in revealing the morphology and functions (genotypes as well as phenotypes) of biological tissues, cells, bacteria and viruses, with impressively high chemical specificity. Despite their prevalence, these molecular-specific contrast agents are not always ideal in view of the complication introduced by cytotoxicity and photobleaching of fluorescence, not to mention the laborious and costly sample preparation procedures associated with staining and labeling.

In contrast, endogenous (intrinsic) parameters, e.g. optical (e.g. light scattering, refractive index), physical (e.g. size, morphology) and mechanical (e.g. mass density, stiffness or deformability, cell traction and adhesion force) properties of the biological specimens, have now been recognized as the new dimensions of phenotypic information, which are valuable in bioassays as complementary to the well-acclaimed molecular-specific information. However, these intrinsic parameters have long been left uncharted, particularly in the context of high-throughput bioassays. Thus, it would be a transformative bioassay technology if one could reveal these intrinsic parameters together with the gold-standard molecular biomarkers—creating a new information/data space for high-throughput and high-content biomedical analysis.

There is a class of microfluidic technologies, broadly named “centrifugal microfluidics,” in which a centrifugal propulsion mechanism is harnessed during spin/rotation for active fluid control and thus sample processing on a chip, e.g. fluid sampling, mixing and valving, and interfacing to external pumps. For example, centrifugal microfluidics enhances antigen binding (affinity) with antibody-coated surfaces in affinity immunoassays. Centrifugal forces also facilitate cell separation and sorting, which have found applications in circulating tumor cell screening. Centrifugal microfluidic technologies have been commercialized for applications including blood parameter analysis, immunoassays and nucleic acid analysis. However, existing technologies lack the ability to deliver ultrafast high-resolution imaging during rotation/spinning operations for real-time high-throughput monitoring (because of the lack of high-speed camera/laser scanning technologies). They also lack the ability to extract the combination of biophysical and biochemical signatures of the biospecimen for high-content analysis, especially in the context of cell-based and tissue-based assays (because they overwhelmingly rely on slow fluorescence imaging, which helps to extract the biochemical information only).

Overcoming the technical and fundamental limitations that exist in traditional imaging methods and hinder the ability to achieve high-throughput and high-resolution imaging bioassay, two new techniques similarly based on the concept of all-optical laser-scanning imaging are developed. One is called “time-stretch imaging”, which is built on temporally stretching broadband pulses by using dispersive properties of light in both spatial and temporal domains. It achieves continuous image acquisition at an ultrahigh frame rate of 1-100 million frames per second. See Lei et al., “Optical time-stretch imaging: Principles and applications,” Appl. Phys. Rev. 3, 011102 (2016); http://dx.doi.org/10.1063/1.4941050, which is incorporated herein by reference in this entirety. Another technique is called “free-space angular-chirp-enhanced delay (FACED) imaging, which is operated based on the use of a pair of quasi-parallel plane mirrors with high-reflectivity (>99%) transforming a laser pulsed beam into an array of spatiotemporally encoded beamlets for laser-scanning. Not only can FACED achieve a line-scan rate as high as 10 MHz similar to time-stretch imaging, but also extended imaging modalities that are impossible with time-stretch imaging, such as bright-field color imaging fluorescence imaging, multi-photon imaging, to name a few. See Jianglai Wu et. al, “Ultrafast Laser-Scanning Time-Stretch Imaging at Visible Wavelengths,” Light: Science & Applications 6, e16196 (2017).

### SUMMARY OF THE INVENTION

The present invention provides advantageous systems and methods for high-throughput multiplexed rotating/spinning multi-scale bioassays, ranging among biomolecules, micro-organisms and cells to tissue/scaffold sections in 2D or 3D.

Embodiments of the invention also involve a technique for performing high-throughput and high-content 2D and 3D imaging bioassays in an ultrafast rotating motion. Many embodiments of the invention feature ultrafast, wide field-of-view (FOV), high-resolution optical laser scanning imaging techniques integrated with a versatile large-format bioassay platform, which supports ultra-large-scale quantitative measurements of a wide-range of biological specimens among biomolecules, microorganisms and cells, to entire tissue sections/scaffolds in 2D or 3D. Operating at the ultrahigh-speed frame-rate based on all-optical laser-scanning imaging (e.g. time-stretch or FACED imaging), many embodiments of the invention achieve the high-throughput measurement (read-out) by ultrafast rotating scanning motion of the bioassay platform or the imaging illumination, at a speed and an FOV that current standard camera/laser-scanning technologies cannot achieve, for capturing microscopic images without motion blur and sacrificing the image resolution. The high-content measurement (read-out) is enabled by extracting not only biomolecular and biochemical information (e.g. assisted by biochemical specific biomarkers), but also an assortment of quantitative parameters normally absent in other bioassay platforms (particularly in the high-throughput systems). These include optical (e.g. light scattering, refractive index), physical (e.g. size, morphology, mass, density) and mechanical (e.g. stiffness or deformability, cell traction and adhesion force) properties of the biological specimens. This is an unprecedented combination of assay throughput and content, which is due to the ultrafast motion together with the ultrafast quantitative high-resolution imaging technique.

Embodiments of the present invention uniquely provide both the large-volume and high-complexity biomedical data—ushering in a paradigm shift in medical science research and clinical diagnostics, which is the current move from hypothesis-based to data-driven biomedicine. The rationale of such a move is that large-scale data not only enables better informed decision making, but also leads to the discovery of new insights. For example, one grand challenge in biology and molecular pathogenesis of disease is to identify rare stem cells/progenitors within an enormous and heterogeneous population. The knowledge of their characteristic signatures (from the cellular to molecular levels) is essential, yet is limited in regenerative medicine. Furthermore, applications can also extend to clinical settings in detecting cells at different stages of differentiation or to quantify rare aberrant cells during early disease processes, especially for rare cancer cell screening. Another example is drug development processes in which there has been a pressing need for highly-multiplexed imaging bioassays (involving cell-based or tissue-based assay) for high-throughput phenotypic drug screening against tens to hundreds of thousands of chemical compounds. Therefore, there is an imminent need for a transformative technology, which can gather high-content data (morphological, phenotypic and molecular) for numerous individual cells and tissues within heterogeneous populations, in order to analyze them at high-speed, and in great detail.

Compared to the conventional bioassay technologies, embodiments of the present invention involve a high-throughput multiplexed bioassay platform based on ultrafast laser-scanning imaging integrated with a high-speed rotating assay substrate or rotating illumination. In addition, the speed and FOV achieved in the imaging assay platform of the present invention cannot be achieved by any existing cameras and laser-scanning/sample-scanning technologies, and can only be made possible with this invention. In many embodiments of the present invention, unidirectional axial scanning during high-speed substrate/illumination rotation allows 3D imaging in real-time. Also, according to the desired applications, the number of imaging FOVs and even the shapes of individual FOVs across the entire platform can flexibly be engineered without compromising the imaging speed and throughput. Note again that the invention shows a versatile bioassay platform compatible with biochemical-specific molecule binding assay, cell-capture assay, cell culture assay and tissue section/scaffold assay. Embodiments of the invention provide a high-content quantitative imaging assay that is capable of simultaneously extracting biophysical (optical, mechanical properties) and biochemical properties, which is also able to integrate with active centrifugal microfluidic technologies for fully automated sample processing, imaging and analysis on the sample assay platform.

Apparatus for carrying out the subject invention utilizes laser pulses from a single or plurality of pulsed lasers, or intensity-modulated continuous wave (CW) lasers. Either one of the two different imaging modalities can be implemented, i.e., (1) time-stretch imaging in which spectral-encoding of the image is involved, or (2) FACED imaging in which no spectral encoding is involved. These pulses are first stretched within a medium (e.g. a dispersive fiber for time-stretch imaging or a quasi-parallel mirror pair for FACED imaging) to form temporal waveforms, which are then guided to the imaging system by a beam splitter. In time-stretch imaging, a holographic diffraction grating together the relay lenses and an objective lens are used to transform the wavelength-swept beams into one dimensionally spectrally-encoded line-scan beams, which are projected onto a modified spinning disk substrate. In FACED imaging, the line-scan beam is directly projected onto the spinning disk substrate. Contact with a specimen on the disk substrate causes the beam to become encoded with an image of the sample or specimen. The line-scan beams encoded with the sample image are returned along the same path, by placing a mirror at the entrance pupil of the back objective lens, thus forming a double-pass configuration. Upon being recombined back into a Gaussian beam profile, the imaged-encoded beams are eventually detected by a high-speed photo-receiver and recorded by a high-speed real-time digitizer and electronic signal processor. Note that the image-encoded line-scan beams can also be re-coupled by lens systems after the back objective lens, which forms a Gaussian beam profile as in the double-pass configuration. The recombined image-encoded beams can also eventually be detected by the high-speed photo-detector. This single-pass transmission configuration is particularly relevant to FACED imaging due to its simplicity in terms of light coupling and light projection without the use of diffraction grating as in time-stretch imaging.

The modified spinning disk (e.g. DVD) assay platform employed in this invention may have four assay wells/sites although they could be any integer number. A sample of the specimen is located in each site. In many embodiments of this invention, the substrate containing the assay sites is composed of two optically transparent layers (e.g. polycarbonate layers obtained from two separate DVDs), which are bonded together with UV-cured adhesive. This creates assay chambers with a height of ˜3-1,000 μm defined by spacers that are also carefully aligned to stabilize the rapid spinning motion.

### BRIEF DESCRIPTION OF THE DRAWINGS OF THE SUBJECT INVENTION

The foregoing and other objects and advantages of the present invention will become more apparent when considered in connection with the following detailed description and appended drawings in which like designations denote like elements in the various views, and wherein:

FIG. 1 is a flow chart of the method of operation for the present invention;

FIG. 2 is a flow chart of a versatile bioassay platform according to the present invention, which is compatible with molecules, cells and tissue sections/scaffolds, and which utilizes modifications of disk substrates for specific capturing, cell culturing and tissue mounting;

FIG. 3A shows the general arrangement of a rotating/spinning platform with astatic line-scan, which is useful with the present invention;

FIG. 3B shows the general arrangement of a rotating/spinning line-scan with a static platform, which can be used with the present invention;

FIG. 3C shows an example of an implementation of a rotating line-scan illumination with a miniaturized optical assembly integrated with a rotating carrier according to the present invention;

FIG. 3D shows an example of an implementation of a rotating line-scan illumination with a miniaturized mirror-based optical assembly integrated with a rotating carrier according to the present invention;

FIG. 4A shows an example of arbitrary field-of-view imaging capability of the system according to the present invention;

FIG. 4B shows an example of an implementation of whole disk imaging using spiral scanning method;

FIG. 4C shows an example of an implementation of whole disk imaging using ring scanning method;

FIG. 4D shows an example of an implementation of an array of segmented field-of-views with reconfigurable areas according to the present invention.

FIG. 5A shows an illustration of an implementation of 3D tissue structure imaging, which is achieved by mounting a 3D tissue block onto a spinning disk substrate. This spinning sample is hence optically sectioned by all-optical laser-scanning imaging along the axial direction, of which the 2D optically sectioned images are then digitally stacked, stitched and reconstructed to a 3D volumetric tissue block structure.

FIG. 5B shows an illustration of an implementation of 3D tissue structure imaging, which is achieved by mounting a 3D tissue block onto a static disk substrate. This static sample is hence optically sectioned by a rotating all-optical laser-scanning imaging along the axial direction, of which the 2D optically sectioned images are then digitally stacked, stitched and reconstructed to a 3D volumetric tissue block structure.

FIG. 5C shows an illustration of an implementation of 3D tissue structure imaging, which is achieved by sectioning a 3D tissue block into multiple tissue slices. The tissue slices are then mounted onto a spinning disk substrate and hence imaged by all-optical laser-scanning imaging, of which the 2D tissue images are then digitally stacked, stitched and reconstructed to a 3D volumetric tissue block structure.

FIG. 5D shows an illustration of an implementation of 3D tissue structure imaging, which is achieved by sectioning a 3D tissue block into multiple tissue slices. The tissue slices are then mounted onto a static disk substrate and hence imaged by a rotating all-optical laser-scanning imaging, of which the 2D tissue images are then digitally stacked, stitched and reconstructed to a 3D volumetric tissue block structure.

FIG. 6A shows a schematic of the DVD imaging cell-based assay system of the present invention, which is based on time-stretch laser-scanning imaging;

FIG. 6B shows a schematic diagram of the substrate of the present invention, which is composed of two polycarbonate layers obtained from two DVD, which are bonded together with UV-cured adhesive;

FIG. 6C shows the image stitching algorithm according to the present invention;

FIG. 6D shows a schematic diagram of the substrate of the present invention, which is composed of one polycarbonate layers obtained from DVD and a glass substrate with tissue sections, which are bonded together with fluorogel;

FIG. 7A shows a 420 μm×34 mm stitched image of MCF-7 cultured on a disc (imaging at 900 rpm (linear speed of ˜4 m/s));

FIG. 7B shows an enlarged view of an area indicated by dashed line in FIG. 7A;

FIG. 7C shows an enlarged view of the upper area indicated by dashed line in FIG. 7B of the stitched image of MCF-7 in FIG. 7B;

FIG. 7D shows an enlarged view of the middle area indicated by dashed line in FIG. 7B in the stitched image of MCF-7 in FIG. 7B;

FIG. 7E shows an enlarged section of the bottom area indicated by dashed line in FIG. 7B stitched image of MCF-7 in FIG. 7B;

FIG. 7F shows a phase-contrast static image of the same area of MCF-7 in FIG. 7C taken by commercial light microscope;

FIG. 7G shows a phase-contrast static image of the same area of MCF-7 in FIG. 7D taken by commercial light microscope;

FIG. 7H shows a phase-contrast static image of the same area of MCF-7 in FIG. 7E taken by commercial light microscope;

FIG. 8A shows a modified DVD assay design for time-stretch imaging of specifically-captured biotinated-polystyrene microparticles;

FIG. 8B shows time-stretch images of captured microparticles in wells T (top) and C (bottom) taken under a spinning speed of 3,000 rpm (speed of 14 m/s);

FIG. 8C shows static images of the wells T (top) and C (bottom) taken by ordinary light microscope;

FIG. 8D shows a statistical size distribution of the captured 4657 microparticles analyzed from the time-stretch image;

FIG. 9A shows a modified DVD assay design for time-stretch imaging of specifically-captured MCF-7;

FIG. 9B shows a time-stretch image of antibody-captured MCF-7 cells in the target well on the spinning DVD (at a spinning speed of 2,400 rpm and a linear speed of 11 m/s);

FIG. 9C shows an enlarged section of the large-area stitched image of MCF-7 in FIG. 9B;

FIG. 9D shows an enlarged section of the large-area stitched image of MCF-7 in FIG. 9B;

FIG. 9E shows an enlarged section of the large-area stitched image of MCF-7 in FIG. 9B;

FIG. 9F shows an image of specific captured MCF-7 cells in the area with only streptavidin coated to be control;

FIG. 9G shows the analysis of the cell-capture specificity in the experiments;

FIG. 9H shows static images of the captured cells (phase-contrast (left) and fluorescence (right)) treated with vital staining (propidium iodide) on the DVD substrate, taken after 2 minutes of spinning;

FIG. 9I shows static images of the captured cells (phase-contrast (left) and fluorescence (right)) treated with vital staining (propidium iodide) on the DVD substrate, taken after 32 minutes of spinning;

FIG. 10A shows a specimen of MCF-7 (25%) mixed with human buffy coat (75%) used for antibody-captured, and thus enrichment of, MCF-7;

FIG. 10B shows time-stretch images of the enriched MCF-7 (with anti-EpCAM antibody) in the target well and in the control well taken at a spinning speed of 900 rpm (linear speed of 4 m/s); and

FIG. 10C shows static images (top: phase contrast; bottom: fluorescence) of the enriched MCF-7 further stained with green fluorescent dye.

FIG. 11A shows stitched time-stretch bright-field images of the human bone tissue.

FIG. 11B shows stitched time-stretch phase image of the human bone tissue.

FIG. 11C shows stitching algorithm implemented for phase images stitching.

### DETAILED DESCRIPTION OF EXEMPLARY EMBODIMENTS OF THE SUBJECT INVENTION

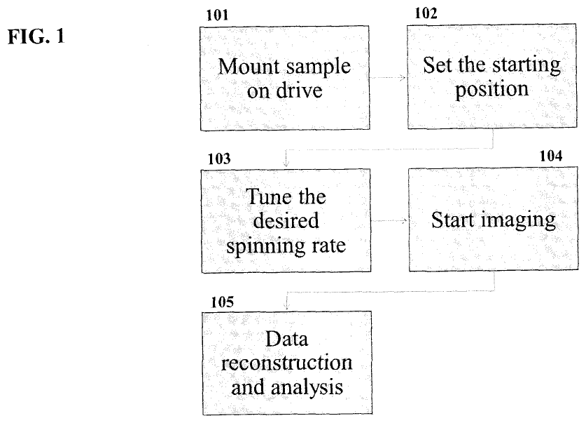

The present invention relates to systems and methods for high-throughput versatile multiscale spinning/rotating imaging bioassays. More specifically, the present invention is embodied in the apparatus, methods and results, as illustrated in FIG. 1 through FIG. 11C. It will be appreciated that the apparatus may vary as to configuration and as to details of the parts, and that the method may vary as to the specific steps and sequence, without departing from the basic concepts as disclosed herein.

The invention can be embodied in the format of an assay with ultrafast spinning/rotating motion. FIG. 1 illustrates that the assay read-out is based on any ultrafast laser-scanning imaging strategy (e.g. time-stretch imaging and FACED imaging) that can surpass the speed limitation of the classical laser scanning technologies (e.g. galvanometric mirrors, rotating polygonal mirrors, acousto-optics deflectors).

The general method for carrying out the present invention involves a first step 101 of mounting a sample of the specimen on a rotatable disk drive as shown in FIG. 1. At step 102 the starting position of the drive is sent and at step 103 the desired spin rate for the disk drive is set. Next at step 104 imaging of the samples on the disk is started, which produces a stream of serial output data. This serial image data is reconstructed and analyzed at step 105 to provide the bioassay.

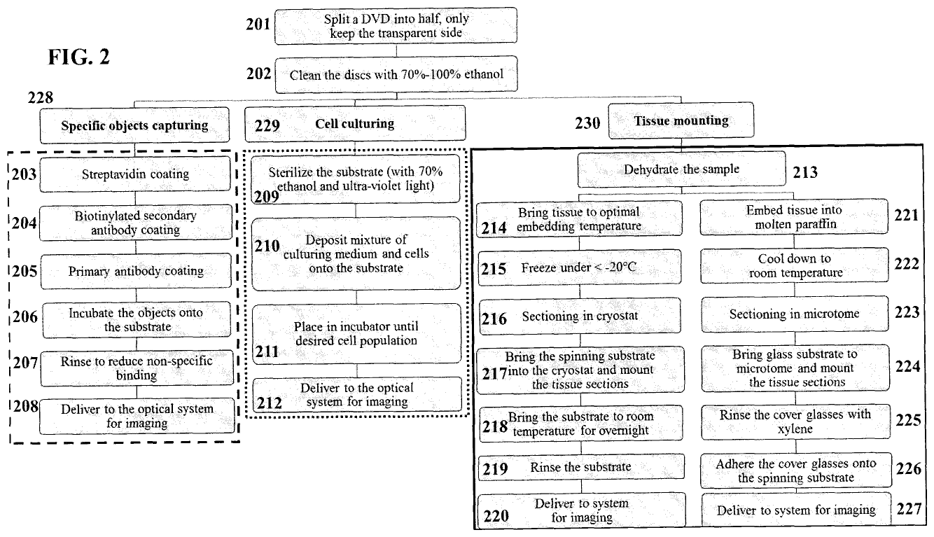

As shown in FIG. 2, this basic method can be implemented with a disk or substrate especially prepared for a particular task such as capturing data from specific objects, cell culturing and tissue mounting. For any of these techniques, the first step 201 is to create the substrate upon which the sample is to be mounted. In many embodiments employing DVD, this is done by first splitting a DVD disk in half and keeping only the transparent half. This transparent disk substrate is cleaned at step 202 with 70% to 100% ethanol.

If the substrate is to be used for capturing specific objects (labelled as 228), the disks are coated with streptavidin at step 203. Next a biotinylated secondary antibody coating is applied at step 204, followed by a primary antibody coating at step 205. The objects to be assayed are placed in wells on the disks and are incubated for a period of time at step 206. After the incubation the disks are rinsed to reduce non-specific binding at step 207. This removes the material that is not at a binding site. Finally the substrate is delivered to the optical system for imaging (step 208) so as to form the bioassay.

When used for cell culturing, the clean disk from step 202 is sterilized, e.g., with 70% ethanol and ultra-violet light at step 209. At step 210 a mixture of culturing medium and cells is deposited onto the substrate. Then at step 211 the substrate is kept in an incubator until the desired cell population is present on the substrate. Finally the substrate is delivered to the optical system for imaging at step 212 so the bioassay can be performed.

Should it be desired to mount tissue on the substrate (labelled as 230), the tissue sample is first dehydrated at step 213. Then at step 214 the tissue is brought to the optimal temperature for embedding it with optimal cutting temperature (OCT) compound. At step 215 the tissue with embedded OCT is frozen to less than −20° C. in a cryostat. While in the cryostat at step 216 the tissue is cut into sections. At the next step (217) the substrate is brought into the cryostat and the tissue sections are mounted on it. The substrate with the frozen tissue is then brought to room temperature over night at step 218. At step 219 the substrate with the mounted tissue is rinsed and at step 220 it is delivered to the optical system of imaging.

Should it be desired to mount tissue (labelled as 230) on the substrate with thinner thickness, the tissue sample is first dehydrated at step 213. Then at step 221 the tissue is embedded into molten paraffin. At step 222 the tissue with embedded paraffin is cooled down to room temperature. While in the cryostat at step 223 the tissue is cut into sections in microtome. At the next step 224 glass substrates are brought into the microtome and the tissue sections are mounted on it. The glass substrates with the frozen tissue are then rinsed with xylene at step 225. At step 226 the glass substrates with the mounted tissue are adhered on the cleaned substrate mentioned in [0064] and at step 227 it is delivered to the optical system of imaging.

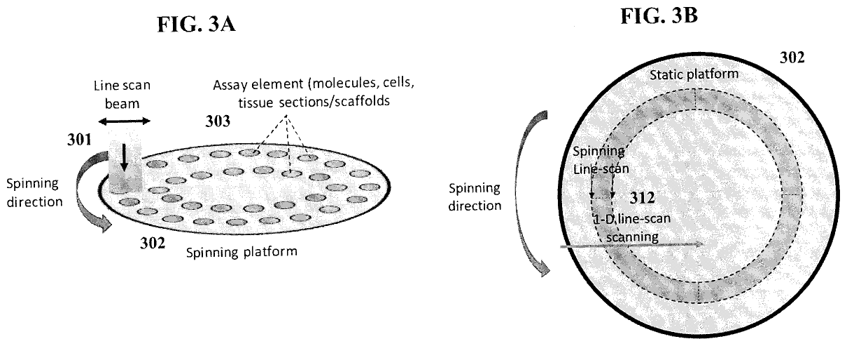

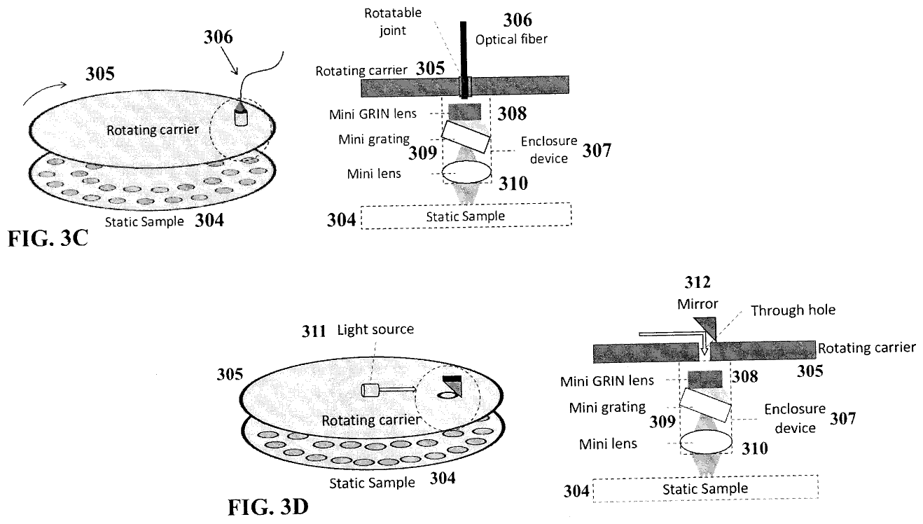

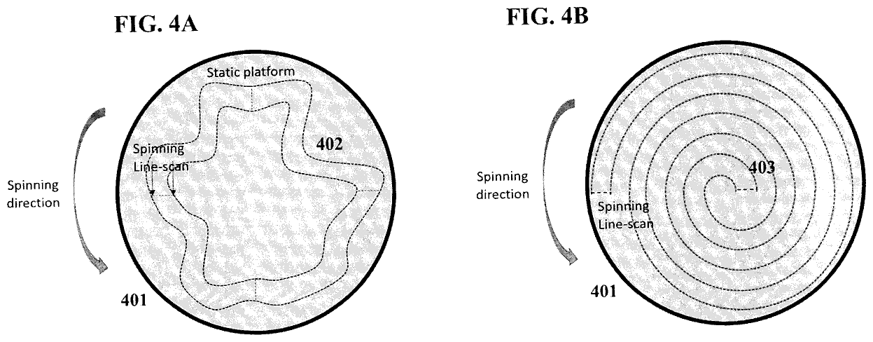

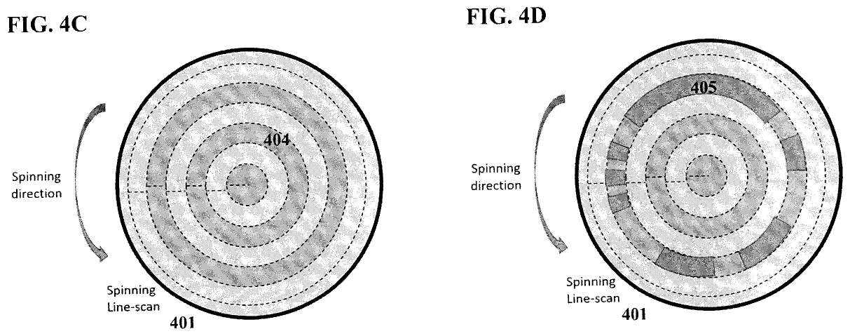

FIG. 3A shows a one-dimensional (1D) line-scan 301 illuminating array elements 303 on a spinning platform or substrate 302. The speed requirement for the 1D line scan has to be beyond 1 MHz in order to accommodate the high-speed motion of the assay (e.g. spinning trajectories). 2D high-resolution images of the specimen (e.g. microscopic images of tissues, cells or microarray of the molecules) are captured by the illumination of the assay sample platform, which has a unidirectional rotating motion, i.e. either to rotate the assay platform as in FIG. 3A, which is imaged by a static line-scan 312 illumination or to rotate/spin the line-scan illumination on the static assay platform as shown in FIG. 3B. When the substrate 304 is static or stationary as in FIG. 3B, the illumination from fiber 306 is rotated by a carrier 305. Note that this unidirectional rotating motion mitigates the mechanical instability and back-lash problem brought on by the conventional strategies of back-and-forth scanning or zig-zag-path scanning. Rotating/spinning illumination is achieved by, but not limited to, the following approach (FIG. 3C): the line-scan optical beam can be directed to an integrated miniaturized optical assembly (consisting of graded-index (GRIN) lens 308, miniaturized relay (Mini grating) lens 309 and objective lens 310) mounted on a rotating carrier 305. These elements are mounted in an enclosure 307. The illumination is provided from optical fiber 306 which is attached to an outer edge of the rotating carrier 305 by means of a rotatable joint. The joint keeps the fiber from twisting while the carrier rotates. In this way the assembly functions as a module that projects the line-scan beam onto the static assay platform. Note that, depending upon the ultrafast line-scan technologies, the line-scan beam can be directed to the assembly through optical fiber 306 or in free-space by bulk optics (FIG. 4A). Note that the illumination elements 301, 306, 311 can also be actuated along the radial direction during spinning in order to access wider 2D FOV.

The FOV of the imaging bioassay system can be arbitrarily engineered. For examples, it can be a continuous FOV covering the entire spinning disk 401, or an array of discrete FOV with different sizes defined by the users, or even the FOV with any arbitrary shapes 402, including but not limited to the line-scan area as in FIG. 4A. This can be controlled by the relative motion between the spinning 403/rotating 404 illumination or the bioassay disk. The scanning for the entire disk can adopt schemes including but not limited to as in FIG. 4B and FIG. 4C. the FOV can also be an array of segmented field-of-views with reconfigurable areas (segments in blue with label of 405) as illustrated in FIG. 4D.

Notably, as the spinning rate of the system can be flexibly tuned from 500 to 25,000 revolutions per minute (rpm), implying an ultra-large-FOV imaging (generally along the circumferential direction) with a video frame rate, i.e. >10 Hz. Again, the effective FOV can be engineered as multiple discrete areas along the circumferential direction. All of these areas can be imaged simultaneously at large-scale (>cm²), and at video rate. This unique capability facilitates real-time video-rate cellular dynamics monitoring in the area of interests, e.g. cell proliferation, cell traction.

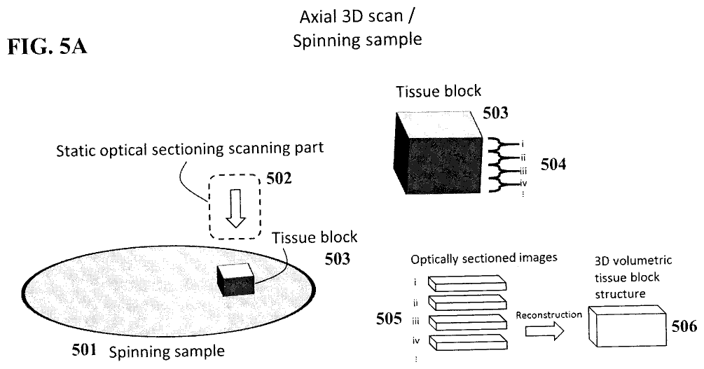

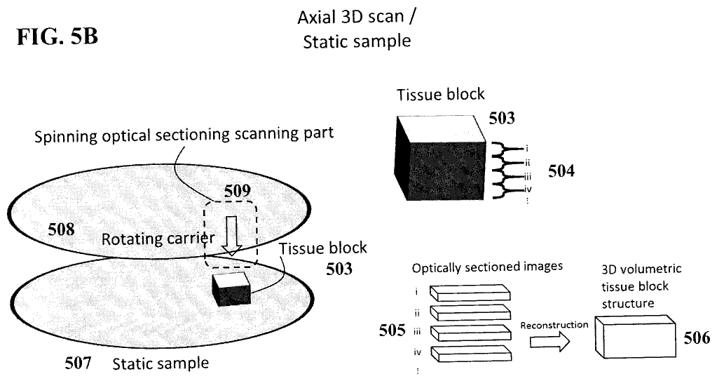

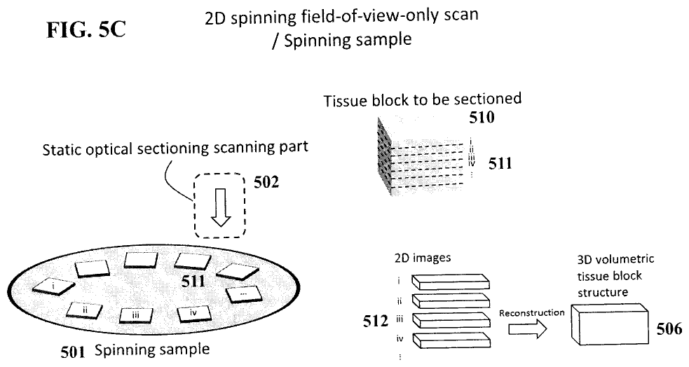

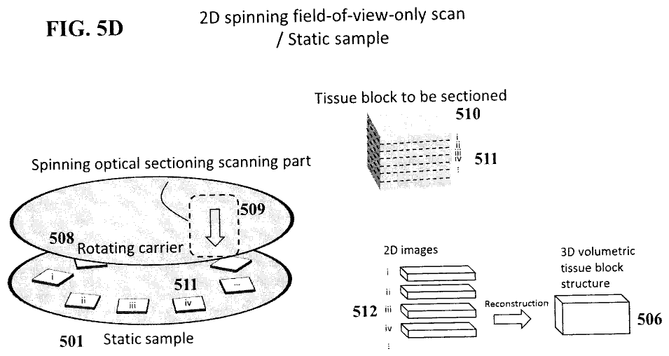

Apart from offering a unique monitoring on 2D bioassays, the current invention can also be extended to 3D tissue structure imaging at an ultrafast rate. FIG. 5 shows the illustrations of how 3D tissue structures can be mounted and imaged by the current invention. This, similar to previous illustrations on 2D imaging, can be performed on both spinning/static disk substrates. In addition, the 3D tissue structures can be treated and imaged for different forms of sample types, e.g. tissue blocks/tissue slices. FIGS. 5A-5B describe how a 3D tissue block 503 can be sectioned optically together with an axial scanning to achieve 3D tissue imaging. FIG. 5A shows an illustration of an implementation of 3D tissue structure imaging, which is achieved by mounting a 3D tissue block 503 onto a spinning disk substrate 501. This spinning sample is hence optically sectioned 504 by all-optical laser-scanning imaging 502 along the axial direction, of which the 2D optically sectioned images 505 are then digitally stacked, stitched and reconstructed to a 3D volumetric tissue block structure 506. FIG. 5B shows an illustration of an implementation of 3D tissue structure imaging, which is achieved by mounting a 3D tissue block 503 onto a static disk substrate. This static sample 507 is hence optically sectioned 504 by a rotating all-optical laser-scanning imaging 508-509 along the axial direction, of which the 2D optically sectioned images 505 are then digitally stacked, stitched and reconstructed to a 3D volumetric tissue block structure 506.

FIGS. 5C-5D, on the other hand, describe how a 3D tissue block can be first sectioned mechanically and then mounted onto the disk substrate to achieve 3D tissue imaging. FIG. 5C shows an illustration of an implementation of 3D tissue structure imaging, which is achieved by sectioning a 3D tissue block 510 into multiple tissue slices 511. The tissue slices are then mounted onto a spinning disk substrate 501 and hence imaged by all-optical laser-scanning imaging 502, of which the 2D tissue images 512 are then digitally stacked, stitched and reconstructed to a 3D volumetric tissue block structure 506. FIG. 5D shows an illustration of an implementation of 3D tissue structure imaging, which is achieved by sectioning a 3D tissue block 510 into multiple tissue slices 511. The tissue slices are then mounted onto a static disk substrate 501 and hence imaged by a rotating all-optical laser-scanning imaging 508-509, of which the 2D tissue images 512 are then digitally stacked, stitched and reconstructed to a 3D volumetric tissue block structure 506.

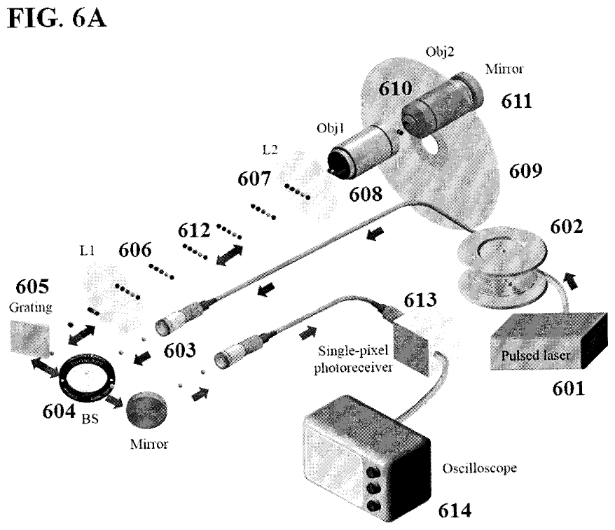

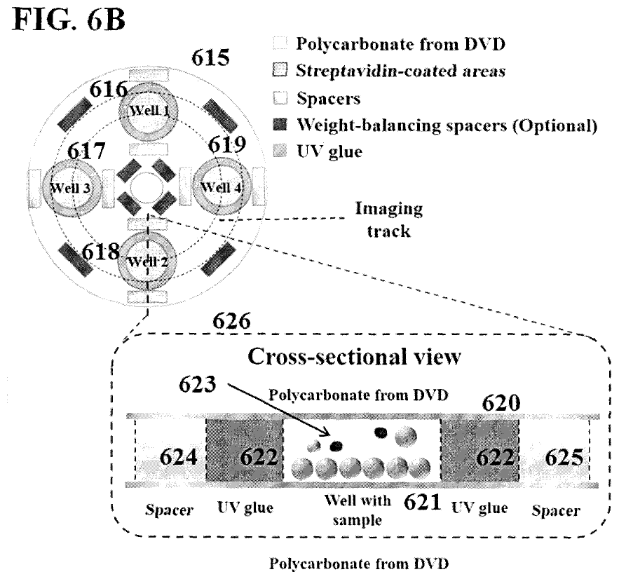

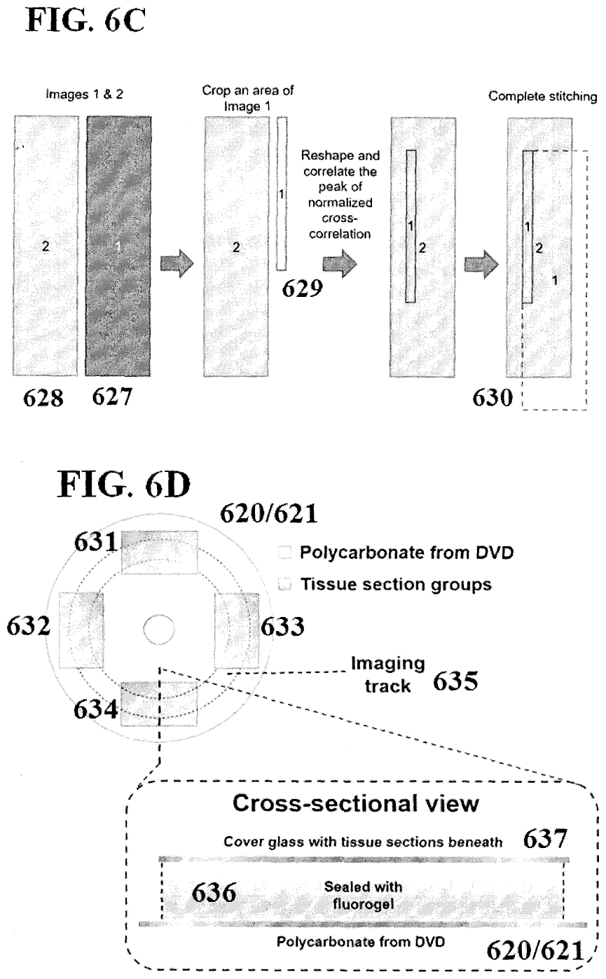

FIG. 6A depicts a device of the subject invention in which laser pulses from a fiber 603 (from a fiber mode-locked laser 601) are first time-stretched within a dispersive fiber 602 and thus form wavelength-swept waveforms, which are then guided to the imaging system by a beam splitter 604 (BS). A holographic diffraction grating 605 together with relay lenses 606, 607 (L1 and L2) and the objective lens 608 (Obj1) are used to transform the wavelength-swept beams into 1D spectrally-encoded line-scan beams 612, which are projected onto the modified spinning DVD substrate 609. The image-encoded line-scan beams are returned along the same path, by placing a mirror 611 at the entrance pupil of the back objective lens 610 (Obj2), forming a double-pass configuration. Upon being recombined back to the Gaussian beam profile, the imaged-encoded beams are eventually directed by a beam splitter and then detected by a high-speed photo-receiver 613 (bandwidth of 12 GHz) and recorded by a high-speed real-time oscilloscope 614 (bandwidth of 16 GHz; sampling rate of 80 GSa/s).

The substrates of commercial DVDs are typically made of polycarbonate, which is a popular choice of material in biomedical applications because of its biocompatibility and its superior mechanical strength. However, the reflective coatings on DVD generally forbid transmission imaging, the imaging configuration adopted in this work (FIG. 6B). To this end, in one exemplary embodiment the assay platform design of the present invention is based on a double-layer polycarbonate substrate, which is obtained from two separate DVDs. Specifically, each DVD was split into two halves, each of which has the same disc shape but a reduced thickness, such that the originally sandwiched reflective layers can be removed. Only the transparent half (ca. 0.6 mm) of the DVD disk is used for further surface functionalization.

FIG. 6B shows a design schematic of the modified DVD assay platform 615 employed in an embodiment of this invention (four assay wells/sites 616-619 are depicted here as an example, which could be any integer numbers). Referring to the cross-sectional view 626 in FIG. 6B, an upper transparent polycarbonate layer 620 is separated from the lower layer 621 by spacers 624, 625. The disks of the substrate are held together by a UV-cured glue or adhesive 622. As a result, after the cell culture or specific cell-capture procedures, UV-cured adhesive is deposited around the cell-specimen sites 623 such that the cells under test were not in contact with the UV-cured adhesive 622 before they are cured. The spacers 624, 625 are made of any solid materials (for example but not limited to glass) and have a height of 3-1,000 μm. They are carefully positioned at various locations on the substrate 621 (as shown in FIG. 6B) such that the weight is evenly distributed across the substrate in order to ensure stable spinning operation.

Substrate 620 is identical to substrate 621, but is non-functionalized. As indicated, it is stacked and glued on top of the functionalized substrate 621 with the spacers. The top substrate 620 is further pressed to ensure complete contact with all the spacers 624, 625. At this point, the double-layer disc 626 is approximately 1.3 mm thick and contains N pre-defined assay compartments (four shown in FIG. 6B). The double-layer disc assembly is exposed to the spatially confined UV light (Thorlabs CS2010) for at least 30 seconds for further curing. The spatially confined UV illumination avoids UV exposure, and thus phototoxicity of the cell specimens within the compartments.

Due to electrical jittering, the images are not taken at the exact spatial position—either horizontally or vertically. On general image stitching in the current invention, a small proportion of images from 2 images are taken for cross correlation before stitching the entire images, which is shown in FIG. 6C. Normalized cross correlation allows infinite stitching iteration and quicker pattern recognition since fewer pixels are taken for correlation calculations. Large arbitrary FOV can be imaged by performing imaging at various spatial locations prior to large-scale image stitching.

FIG. 6D shows substrate design for tissue sections adhesion. Only a single-layer polycarbonate substrate (620/621) is used. Tissue sections are adhered on cover glass 637 before being sealed with mounting medium 636, e.g. Fluorogel. Multiple tissue sections (631-634) can be adhered onto the same cover glass such that tens of tissue sections can be handled on a polycarbonate substrate. For substrate to be used in tissue imaging, the substrate 620/621 is processed through steps 201-202 in FIG. 2 only.

The present invention can be used for cell culture experiments. When this is the case, the polycarbonate substrate is cleaned and sterilized with 70% ethanol and ultra-violet (UV) light. See steps 209-212 in FIG. 2.

One type of cell culture experiment can be performed with the present invention on the human breast adenocarcinoma cell lines (MCF-7). In such a case the cell line is trypsinized from a culture dish and centrifuged before mixing with standard cell culture medium formulated with 90% Minimal Essential Medium (MEM), 10% Fetal Bovine Serum (FBS) and 1% Penicillin-Streptomycin (Pen Strep). Cells of this type are cultured in a CO₂ incubator and the medium is renewed two to three times per week. See steps 209-212 in FIG. 2.

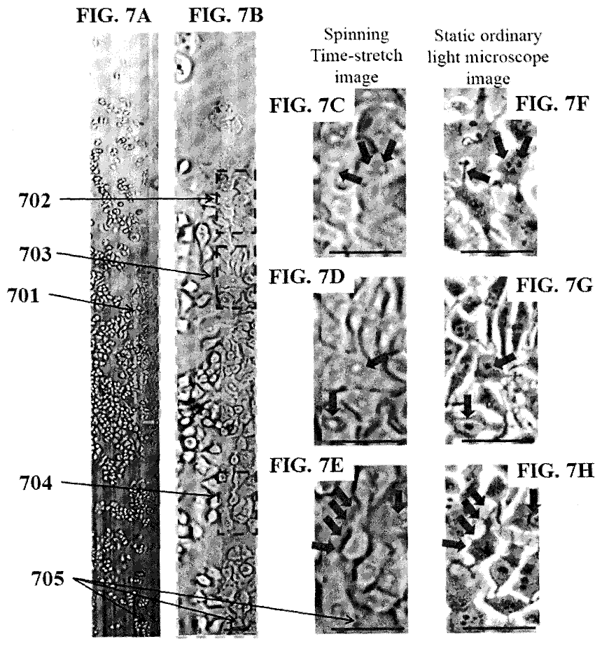

The results of tests with this cell culture are shown in FIG. 7. For these tests, about 30,000 MCF-7 cells were mixed with 300 μL standard cell culture medium and were then loaded on the pre-defined areas on the half-disc substrate. The mixture was spatially confined within the area by surface tension on the hydrophobic polycarbonate surface. This substrate was then incubated for two days before being bonded with another non-functionalized polycarbonate half-disc.

As a proof-of-concept experiment of the subject invention, time-stretch imaging of these MCF-7 adherent cells is performed on the modified DVD at a spinning speed of 900 rpm, which is equivalent to a linear speed of 4 m/s within the line-scan region. Not only did the system capture a large FOV, (as large as 34 mm×420 μm (FIG. 7A)) at a line-scan rate of 11 MHz, but it also delivered high-resolution cellular imaging that reveals sub-cellular structures without motion-blur. FIG. 7B in an enlargement of the area indicated by reference 701 in FIG. 7A. FIGS. 7C, 7D and 7E represent further enlargements of the areas indicated by references 702, 703 and 704, respectively. The lines indicated by reference 705 in FIG. 7 represent 50 μm.

Note that the image contrast can be further enhanced by accessing the phase contrast through the use of interferometry, or phase gradient contrast by an asymmetric-detection technique. Notably, both imaging schemes can further quantify the phase information of cells, from which a set of biophysical phenotypes can be extracted, e.g. cell size, mass, and density.

It was found that the spinning speed regime adopted in this invention can ensure no observable change in morphology of the adherent cells under the ultrafast spinning action. This can be verified by the static images of the same area taken by a conventional light microscope using a 10× objective lens (Nikon Eclipse Ni-U). The cellular morphology visualized in the static images and the time-stretch spinning images are generally consistent with each other (FIGS. 7F, 7G, 7H). The arrows in each of FIGS. 7C, 7D, 7E indicate the key cellular features identified in both time-stretch images and light microscope images of FIGS. 7F, 7G, 7H. In the current system, the FOV of the time-stretch image is limited by the finite memory depth provided by oscilloscope. When integrated with high-throughput data acquisition platform, e.g. graphic processing unit (GPU) or field programmable gated array (FPGA), whole disc imaging in real-time is feasible.

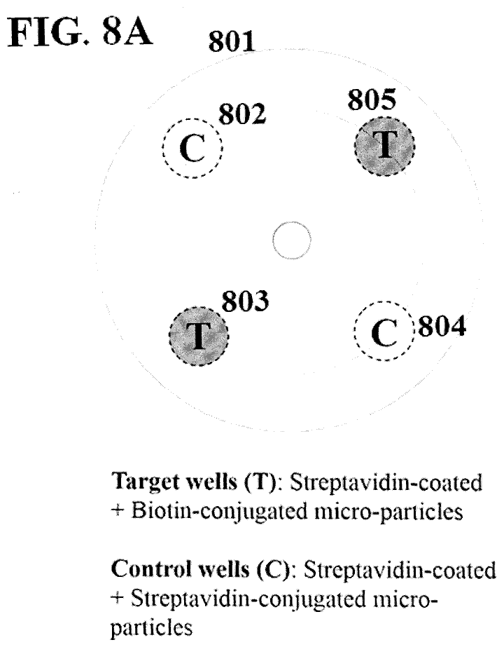

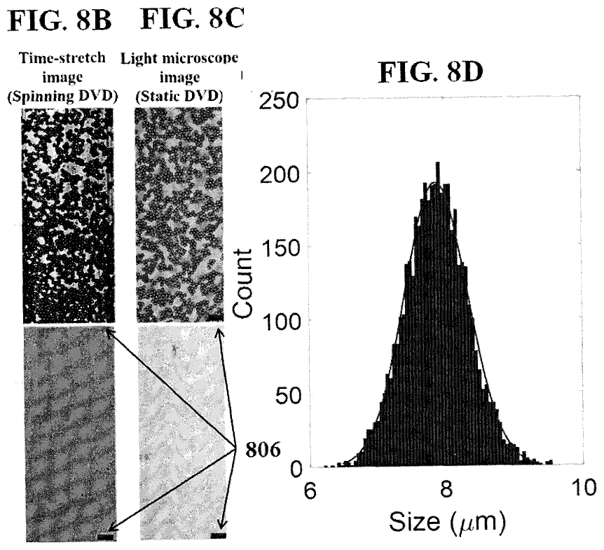

For the test of chemically-specific microparticle-capture, biotinylated polystyrene microspheres (Spherotech, 7.79 μm) may be employed. Then 20 μL of stock supplied microsphere solution is incubated on all pre-defined capture (target) wells of the disc for 30 minutes (see the disc schematic shown in FIG. 8A). All wells are washed with 1× phosphate-buffered saline (PBS) 5 times to prevent non-specific microsphere capture. Gently washing the sites with reverse osmosis (RO) water once for 5 seconds could prevent crystallization of PBS upon drying.

The results for chemically-specific-cell/microparticle-capture experiments are shown in FIGS. 8A-10C. As shown in FIG. 8A, the substrate 801 is further processed with streptavidin coating separately in N pre-defined areas (4 are illustrated as an example in the embodiment of FIG. 8A), which later form the assay wells 802-805. Secondary biotinylated antibodies can be incubated in these areas 803, 805 for further specific capture. See steps 203-208 of FIG. 2. The curve line on the DVD in FIG. 8A indicates the recording area.

The four sites/wells are symmetrically distributed on the disc substrate 801. Two of them 803, 805 are coated with streptavidin for biotinylated-microsphere binding and are labelled T, whereas the other two wells 802, 804 are without streptavidin coating and are defined as control wells marked C (FIG. 8A). For the sake of imaging demonstration and simplicity, a single-layer substrate design and reflection imaging are used in this experiment. This was achieved by removing the back objective lens 610 and the mirror 611 such that only the reflected and back-scattered light is collected. This arrangement was selected because, in contrast to biological cells, polystyrene microspheres give sufficiently high back-scattered light contrast and can be exposed to air during imaging without introducing any detrimental effect to the microspheres. The substrate 801 was dried inside a desiccator prior to high-speed spinning for time-stretch imaging. A stitched image with large FOV of 0.384 mm×140 mm was transformed into a curved image, with an arc of 180° (See the overlaid image on the disc schematic shown in FIG. 8A). Specifically captured microspheres (4,657 microspheres) are clearly visualized under a high rotational speed of 3,000 rpm, or a linear speed of ˜14 m/s (FIG. 8B top), in obvious comparison with the image taken in the control sites, i.e. no microspheres are observed (FIG. 8B bottom). Note that the time-stretch images of the spinning substrate are highly consistent with the static images of the same regions, captured by an ordinary light microscope (FIG. 8C). All scale bars indicated with reference number 806 represent 50 μm. To further exemplify the capability of quantitative analysis derived from this high-throughput imaging technique, individual microspheres were digitally segmented in the image and quantified. The statistical distribution of the size (FIG. 8D) is a Gaussian curve. The measured mean diameter, i.e. 7.85 μm (a standard deviation of 0.68 μm), is consistent with the specification provided by the supplier.

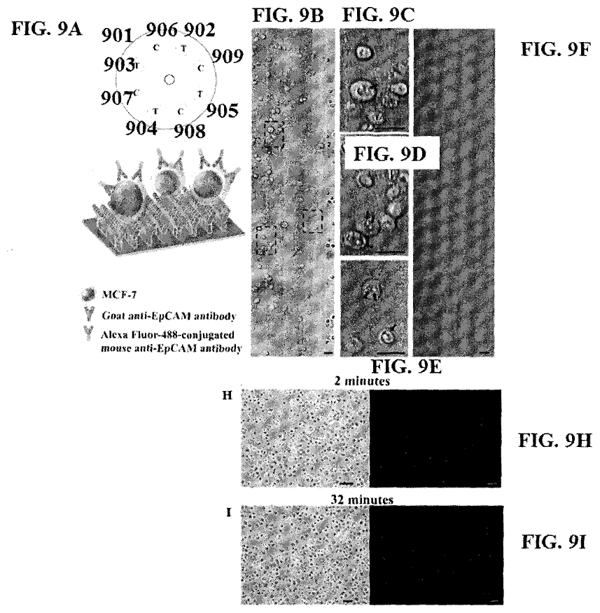

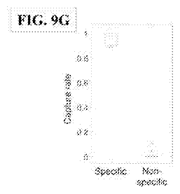

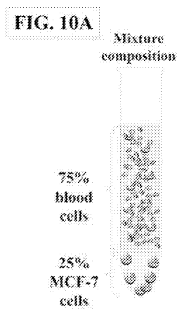

For the tests of chemically-specific cell capture, the results of which are shown in FIG. 9, four or eight target wells are defined on the single-layer polycarbonate substrate and are treated with streptavidin coating (following the protocol provided with BioteZ Polystreptavidin R Coating Kit) (FIG. 9A). Biontinylated horse anti-goat antibody (Vector Labs BA-9500, 10 μg/mL) was incubated in the streptavidin-coated sites for 30 minutes, followed by rinsing 5 times with 1×PBS. Then, goat anti-epithelial cell adhesion molecules (EpCAM) antibody (RnD AF960, 10 μg/mL) was further incubated only in the target wells for 30 minutes and followed by rinsing 5 times with 1×PBS, such that the target wells can capture MCF-7, which has EpCAM as surface markers. See steps 203-208 of FIG. 2.

Next 10 μL MCF-7 in 1×PBS was loaded to all the wells on the substrate for 30 minutes, which allowed for binding between the antibodies and EpCAM, and thus capture of MCF-7 (FIG. 9A). This was followed by rinsing with 1×PBS 5 times so as to reduce non-specific binding. All the coating layers were separately tested and verified by standard biochemical methods. The streptavidin layer was tested with 3, 3′-diaminobenzidine (DAB) staining using biotinylated horseradish peroxidase H (Vector Labs PK-6100) such that the streptavidin-coated substrate appeared to be brown in color upon staining. The biotinylated horse anti-goat antibody (Vector Labs BA-9500) layer on top of streptavidin layer was verified by fluorescence imaging after incubation with the extra Alexa Fluor 488 goat anti-mouse antibody (Life Technologies A-11001) on the substrate (FIG. 9A). For every layer, control experiments were conducted to verify minimal non-specific binding.

The substrate adopted an eight-well design 901: four are target wells coated with anti-EpCAM antibody (marked as T) 902-905 whereas the other four are control wells with only streptavidin coating (marked as C) 906-909. A schematic of the antibody-captured MCF-7 in the target wells is also shown.

The acquired images can be stitched using algorithm that is designed for handling images of giga-pixels in a spinning motion. The algorithm is different from panorama algorithm by not using feature-recognizing algorithm, i.e. scale-invariant feature transform algorithm. The algorithm is based on normalized cross-correlation, which compares trimmed areas from 2 images before calculation. During the calculation of correlation, the images are simultaneously reshaped to compensate the image deformation under high-speed spinning. The position and deformation coefficient where maximum correlation occurs are recorded and the final image can thus be reconstructed, as illustrated in FIG. 6C. Such algorithm is also able to monitor the jittering in rotational rate.

Under the rotational speed of 2,400 rpm (i.e. a linear speed as high as 11 m/s), the time-stretch imaging system of the present invention was able to acquire high-resolution images of individual captured MCF-7 cells in the target wells (FIG. 9B). Note that the final stitched image has a FOV of 0.55 mm×70 mm, covering both the control and target wells (across an arc of 90° as shown in FIG. 9A). The DVD surface modification procedure and the binding specificity are clearly validated by the comparison between the images of the target and control wells (FIG. 9B compared to FIG. 9F). The scale bar at the bottom represents 50 μm. FIGS. 9C-9E show enlarged sections of the areas in dotted line in FIG. 9B.

The specific capture rate was determined to be ˜95.8% whereas the non-specific capture rate was ˜1.4% (FIG. 9C). It should be noted that the specific capture rate is in principle limited by the available binding area. The significance of this demonstration is that time-stretch imaging integrated with this spinning cell-based assay format can reveal not only the morphological information of the cells, but also the biomolecular signature of the cells through biochemically-specific binding (e.g. the surface markers EpCAM of MCF-7 in this case)—important additional information for enhancing the assay accuracy and specificity.

FIG. 9G shows the analysis of the cell-capture specificity in the experiments. The percentages of specificity and non-specificity are calculated from the number of remaining cells upon rinsing out of the number of MCF-7 captured in the antibody-coated and streptavidin-coated wells respectively.

FIG. 9H shows static images of the captured cells (phase-contrast (left) and fluorescence (right)) treated with vital staining (propidium iodide (PI)) on the DVD substrate, taken after 2 minutes of spinning. FIG. 9I shows static images of the captured cells (phase-contrast (left) and fluorescence (right)) treated with vital staining (PI) on the DVD substrate, taken after 32 minutes of spinning.

The viability of the cells tested was also assessed by the high-speed spinning operation of the present invention. Vital staining was performed by incubating captured cells with PI on the substrate. Orange-red fluorescence emission from PI serves as the indicator for dead cells. No noticeable change in the viability of the cells after the 2-min and 32-min spinning operations was observed (only 0.5% increase in the dead cell counts). It verifies that the high-speed spinning during time-stretch imaging introduces minimal detrimental effect to the cells. In addition, the vast majority of the captured cells remained unchanged in their position on the disc over the spinning duration of 32-min (FIG. 9I). It demonstrates the superior binding strength, and thus robustness of this cell-capture assay format.

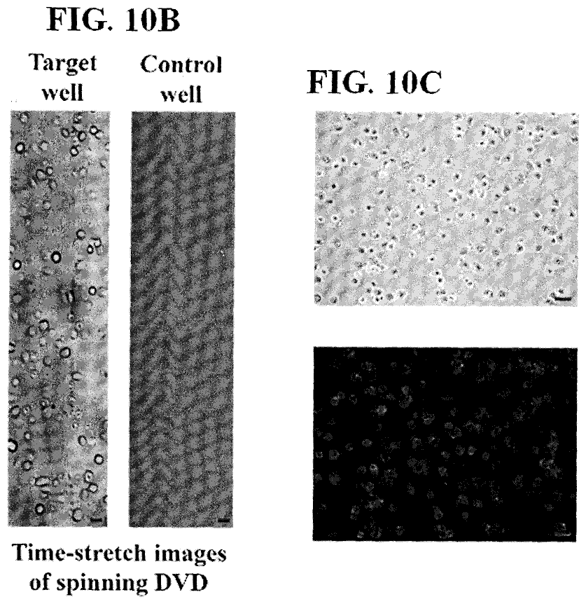

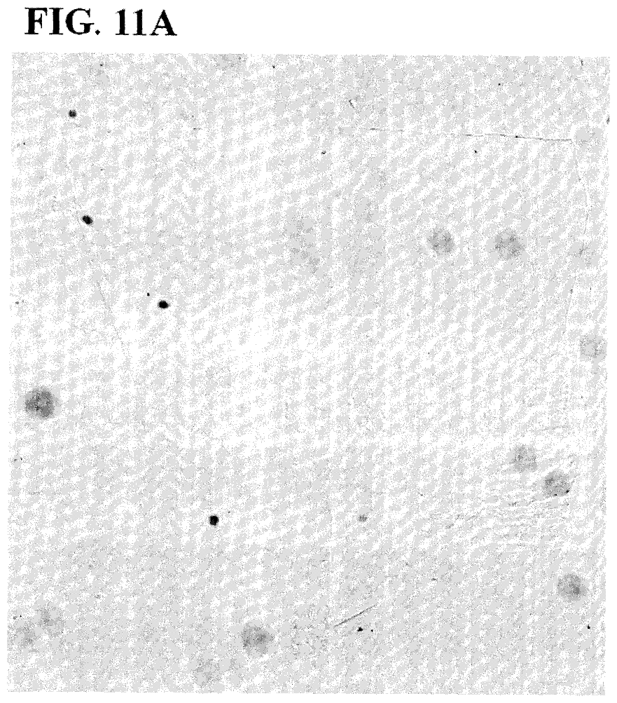

Instead of using pure population of MCF-7 (as shown in FIG. 9), a further experiment was conducted with a mixed population of human blood cells and MCF-7. Specifically, MCF-7 cells were mixed with human buffy coat (extracted from human whole blood) (FIG. 10A), followed by MCF-7 capture and screening on the spinning disc. The same substrate design as shown in FIG. 7A was employed. Similar to the experiment with the pure MCF-7 population (FIG. 9), four out of eight sites 902-905 on disc were designated as target wells, which were coated with streptavidin, biotinylated horse anti-goat antibody and finally goat anti-EpCAM antibody. The screening/enrichment process demonstrated a highly efficient specific cell-capture, which was again visualized by time-stretch imaging, under the spinning speed of 900 rpm (FIG. 10B left). The specificity of MCF-7 capture was further validated by conjugating additional green fluorescent probe (Alexa Fluor-488) with anti-EpCAM antibody and detecting the corresponding fluorescence emission after the time-stretch spinning imaging operation (FIG. 10B right)—confirming the captured cells are not the white blood cells. All scale bars represent 50 μm.

For the tests of MCF-7 enrichment/screening, the buffy coat samples were obtained one day prior to the experiments and were stored at room temperature overnight. On the other hand, MCF-7 cells were trypsinized from the culture dish and were counted such that 600,000 MCF-7 cells were then mixed with 3,000,000 cells from human buffy coat (220 μL in total). Then 10 μL of the mixture solution was incubated in each target site for 30 minutes, followed by multiple disks rinsing (˜7 times) until the blood clot was completely eliminated. The rinsing should be conducted more than 1 time after calibration.

FIG. 10C shows static images (top: phase contrast; bottom: fluorescence) of the enriched MCF-7 further stained with green fluorescent dye. (Alexa Fluor-488 anti-EpCAM (RnD FAB9601G, 10 μg/mL)). This additional staining step was performed to further confirm the MCF-7 enriched on the DVD.

This test is particularly relevant to the applications of CTCs enrichment, detection and enumeration. In this example, we utilized EpCAM, a cell adhesion molecule commonly expressed on epithelial cells, as a biomarker to distinguish MCF-7 cells from the white blood cells through an immunological binding approach. Therefore, the embodiments of the subject invention not only can perform EpCAM-based CTC enrichment, similar to the existing techniques, but also allow in-situ quantitative image analysis of the captured cells with the single-cell precision, thanks to the high-resolution and high-throughput imaging capability. Notably, coupled with quantitative phase time-stretch imaging, this high-throughput spinning imaging cell-based assay could allow single-cell biophysical phenotyping, e.g. cell size, mass, density and other cellular mechanical properties. These intrinsic phenotypes are known to be closely correlated with the malignancy transformation and are thus effective biomarkers of cancer screening as well as drug development. Especially regarding the drug development process, a large-format spinning disc also favours highly-multiplexed imaging assay and can potentially be used for efficient (cancer) drug screening against hundreds to tens of thousands compounds.

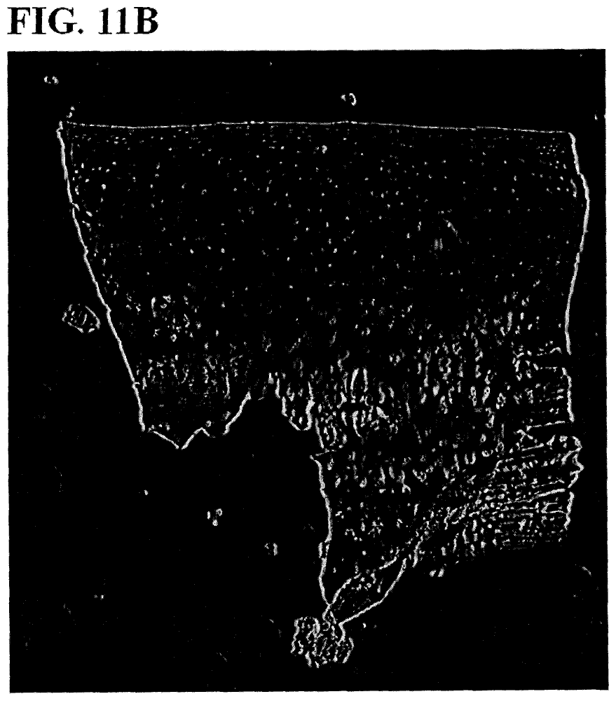

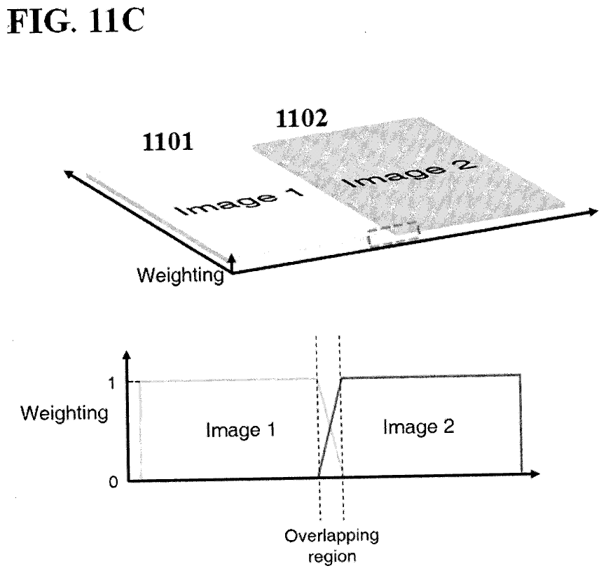

FIG. 11 shows another implementation of label-free large-FOV tissue-section imaging (human cartilage tissue section). In this case, both bright-field and quantitative phase images were captured during spinning (at 2,400 rpm). After that, bright-field images were used for pattern recognition and stitching. The same coordinates for stitching will be used to stitch the corresponding phase images. This reduces the overall computational time for processing the stitching of both bright-field and quantitative phase images (FIGS. 11A and 11B). For the phase image stitching, the overlapping area between 2 images 1101-1102 are weight-averaged using the scheme illustrated in FIG. 11C. Since the reconstructed phase values can have increasing error towards the 2 edges of the image due to optical aberration and low spectral power, this method can cancel out a portion of the error.

The assay format of the subject invention is highly versatile. The rotating planar platform represents a versatile assay format that can be functionalized for a wide range of applications, such as but not limited to, adherent cell cultures, biochemically-specific cell capture, bimolecular affinity assay, 2D tissue section, 3D tissue scaffold, and 3D tissue specimens with tissue clearing techniques. The centrifugal action of the rotating platform can also be harnessed for biomechanical measurement of cells (e.g. cell traction force, cell adhesion force, cellular stiffness), which has not yet been demonstrated in any prior assay technologies. Note the biomechanical properties of cells and tissues have long been known to be closely linked to genetic/epigenetic signatures and thus represent valuable intrinsic biomarkers for cell biology studies and cancer screening, as well as assessments of drug response during drug development process.

In many embodiments, biomechanical measurement of cells can be implemented on the rotating or static substrate 302, 304 (as shown in FIGS. 3A and 3B) which is modified to be compatible with the common traction force microscopy configurations, but under the high-speed spinning/rotating action. This implementation could help visualize in real-time the spatial distribution of the cell traction, adhesion force at the single-cell precision and at high-throughput. In this case, the substrate 302, 304 includes an elastic layer with fiducial markers (e.g. fluorescence beads) at the highest possible density. Laser-scanning imaging on this spinning platform is employed to track their movement and quantify the displacement field from which the single-cell traction force can be evaluated.

On the other hand, the cell stiffness could be inferred by direct imaging (e.g. bright-field and quantitative phase imaging modality) of cell deformation induced by the shear force on the rotating or static substrate 302, 304 under the centrifugal action.

In contrast to the conventional raster scanning of a sample platform or a laser-beam for imaging, which suffer from a slow scanning rate and mechanical back-lash, the present invention relies on high-speed unidirectional rotation, and thus stable sample or illumination scanning at >1,000 rpm. This feature of this high-speed rotating motion mandates the ultrafast laser scanning technology that could achieve a continuous line-scan rate beyond 10 MHz in order to ensure high-resolution imaging free of motion-blur. This explains why almost none of the existing assay techniques based on centrifugal platform is able to incorporate imaging capabilities, i.e., because of the fundamental limitation of the current camera technologies.

To fully understand most if not all cellular signatures, a multimodal imaging platform is usually incorporated. This assay platform is capable of delivering ultrafast laser-scanning quantitative phase imaging (for label-free biophysical phenotyping of cell/tissue; for label-free read-out of biomolecular binding—for affinity assay, e.g. immunoassay) as well as laser-scanning fluorescence imaging or detection (for biochemical phenotyping or read-out of molecules, cells, or tissue). The imaging capability is made possible with time-stretch imaging or FACED imaging.

To this end, the subject invention features an all-optical ultrafast laser-scanning imaging (i.e. FACED imaging or time-stretch imaging) that is believed to be the only available imaging technology capable of delivering quantitative phase and florescence imaging at a line-scan rate beyond 10 MHz. This ultrafast, multimodal imaging capability is achieved within a unified system that allows simultaneous biophysical and biochemical measurements from the biological specimen in real-time, continuously. This is a feature generally absent from any existing assay technologies. Moreover, leveraging the continuous rotating imaging operation, wide-FOV three-dimensional (3D) imaging of cell and tissue specimens can be achieved by continuous unidirectional axial translation of the scanner device or the sample platform. Again, this capability is scarce in assay technologies. This unidirectional spinning and axial translation approach resolves the common backlash problem, which occurs in virtually all galvanometric scanning mirrors, or bulky sample scanning stage in classical automated microscopes, and thus improves the scanning precision for long-term continuous scanning operation.

This invention is also widely compatible with biomolecular affinity assay (e.g. ELISA/EIA), adherent 2D or 3D cell culture, biochemically-specific cell capture assay, WSI, TMA formats and 3D tissue specimens. Based on the integration of the high-speed rotating motion assay strategy and ultrafast all-optical laser-scanning technology, not only can the present invention significantly enhance the assay throughput and content in the current assay applications, e.g. ELISA/EIA, phenotypic drug screening using cellular imaging, ultrahigh-throughput WSI or TMA imaging, the invention also opens new types of imaging assay which are otherwise impossible with the existing assay by harnessing the centrifugal action, e.g. ultra large FOV monitoring of cellular biomechanics (under centrifugal action) at the single-cell precision; real-time monitoring of cellular/molecular affinity kinetics (manipulated by the centrifugal force) at high image resolution.

High-density assay and array matrix on large-area spinning/static platforms can readily be designed and fabricated with existing microfabrication technologies and thus allows highly multiplexed assays at high-throughput.

The present invention also incorporates the established centrifugal microfluidic technologies for active fluid control, e.g. sampling, mixing and valving, on the same disc. Such assay integration allows more advanced assay functionalities and fully-automated workflow (from sample loading, processing and monitoring to analysis) on the sample assay platform. As a consequence, the embodiment of the subject invention represents a unique and versatile assay approach for high-throughput screening in drug screening development, routine pathological assessment, cancer screening and so on.

Apart from the understanding of cellular or biomolecular signatures, the subject invention can be incorporated into tissue sections/scaffolds. The procedures for tissue mounting are illustrated, but not limited to, the following, and which consist of two major steps: (1) a standard cryo-embedding and a loading process of sections onto the substrate in the subject invention. A fresh tissue/scaffold sample is frozen under −20° C., which is prepared for trimming the sample into dimensions matching the support. The trimmed tissue block is then carefully placed at the center of the support (designed for a cryostat), followed by pouring OCT into the support at room temperature. Afterwards, the support with the content is frozen under −80° C. until the block is completely hardened. Before the second step, the block is transferred quickly to a cryostat for cutting.

To load the sections onto the substrate of the present invention, a transparent/reflective substrate (e.g. a DVD) is prepared and cleaned with 70% ethanol. The DVD can be optionally treated to exhibit hydrophilic property. Notably, there are several ways for enhancing hydrophilic behavior on hydrophobic polycarbonate surface, including corona (air) plasma discharge, ozonation, flame plasma discharge and chemical plasma discharge. While all of them serve a similar purpose, i.e., cleaning the surface, they rely on different mechanisms and therefore have different drawbacks.

Corona plasma discharge requires a vacuum condition with high electrical potential difference to discharge an electric arc onto the sample. It requires a specific chamber and careful manual inspection for plasma generation. The treated substrate exhibits good hydrophilic property for a short period of time (less than 5 minutes). While all other methods also require specific tools not easily accessible by the public, these methods are mainly adopted in industry.

Notably flame plasma discharge is generally relatively stable upon aging due to the extensive oxidation by reactions with OH radicals in the flame. The embodiments of the subject invention are based on combining and combusting flammable gas and atmospheric air within the intense blue flame region. Instead of requiring complicated instruments, the present invention generates such intense blue flame with pressurized liquefied butane gas operated with a portable combustor. Due to the relatively low melting point of the DVD, the contact between the blue flame and the DVD must not be continuous. The present invention adopts a repeated zig-zag scanning path for the blue flame contact. A large-area, flat, heat-conducting metal is placed beneath the DVD to ensure rapid heat dissipation. The scanning should be performed for 5 times. The hydrophilic enhancement has been shown to last for weeks.

After treating the substrate DVD with 70% ethanol, the cut tissue sections (at most N consecutive sections) are transferred onto the DVD. When the DVD is full of samples, it is brought to room temperature and the section attachment is allowed. The DVD is then washed with RO water or PBS to clear OCT. Optional staining or other optical modifications can also be performed on DVD.

While the present invention has been particularly shown and described with reference to preferred embodiments thereof; it will be understood by those skilled in the art that various changes in form and details may be made therein without departing from the spirit and scope of the invention.

1. Apparatus for carrying out a multiplexed rotating imaging bioassay, comprising:
a laser generating laser pulses for all-optical laser-scanning imaging;
a modified spinning disk substrate on to which the beams are projected, said substrate having at least one assay well located on it, which well contains a specimen sample;
a back objective lens for receiving the beams from the disk substrate, which have been encoded with information from the sample to form image encoded beams;
an image coupling module for directing the encoded beams onto a beam splitter with a recombined beam profile;
a high-speed photodetector receiving the return beams from the beam splitter;
and a high-speed real-time data recorder that records the output of the photodetector.

2. Apparatus for carrying out a multiplexed rotating imaging bioassay, comprising:
a laser generating laser pulses for all-optical laser-scanning imaging;
a modified static disk substrate on to which spinning illumination beams are projected, said substrate having at least one assay well located on it, which well contains a specimen sample;
a back objective lens for receiving the beams from the disk substrate, which have been encoded with information from the sample to form image encoded beams;
an image coupling module for directing the encoded beams onto a beam splitter with a recombined beam profile;
a high-speed photodetector receiving the return beams from the beam splitter;
and a high-speed real-time data recorder that records the output of the photodetector.

3. The apparatus of claim 1 wherein all-optical laser-scanning imaging including time-stretch imaging, which comprises
a dispersive fiber in which the laser pulses are first time-stretched to form wavelength-swept waveforms;
a beam splitter that directs the wavelength-swept waveforms to an imaging system;
a holographic diffraction grating together with relay lenses and an objective lens forming the imaging system, said imaging system transforming the wavelength-swept waveforms into one dimensionally spectrally-encoded line-scan beams.

4. The apparatus of claim 1 wherein all-optical laser-scanning imaging including FACED imaging, which comprises
a plane mirror-pair with high reflectivity in which the laser pulses are transformed into an array of spatiotemporally encoded beamlets;
a beam splitter that directs the beamlets to an imaging system including relay lenses and an objective lens, said imaging system transforming the beamlets into one dimensionally line-scan beams.

5. The apparatus of claim 1 wherein the disk substrate is composed of two transparent polycarbonate layers obtained from two separate disk substrates, which are bonded together with UV-cured adhesive.

6. The apparatus of claim 1 wherein the disk substrate further includes spacers which determine the height of the spacing between the polycarbonate layers so as to form assay chambers, said spacers being substantially aligned to stabilize the rapid spinning motion.

7. The apparatus of claim 5 wherein the assay chambers have a height of about 3-1,000 μm defined by the spacers.

8. The apparatus of claim 1 wherein the disk substrate has at least four assay wells.

9. The apparatus of claim 1 wherein the image coupling module with an imaging configuration including time-stretch imaging and FACED imaging, which consists of a mirror at the entrance pupil of the back objective lens that reflects said encoded beams so that they return along the same path through the disk substrate and the imaging system so as to form a double-pass configuration.

10. The apparatus of claim 1 wherein the image coupling module with an imaging configuration including FACED imaging, which consists of lens systems after the back objective lens that guide the encoded beam onto the detection light path so as to form a single-pass configuration.

11. The apparatus of claim 1 wherein the modified spinning disk substrate includes an assay well compatible with adherent cell culture.

12. The apparatus of claim 1 wherein the modified spinning disk substrate includes an assay well compatible biochemically-specific cell-capture.

13. The apparatus of claim 1 wherein the modified spinning disk substrate includes an assay well compatible with tissue specimens including 2D and 3D tissue structures.

14. The apparatus of claim 1 wherein the modified spinning disk substrate can be spun and imaged by all-optical laser scanning imaging to generate an arbitrarily-shaped field-of-view;
a spiral scanning field-of-view;
or a ring scanning field-of-view;
or an array of segmented field-of-views with reconfigurable areas.

15. The apparatus of claim 13 wherein the modified spinning disk substrate for mounting 3D tissue specimens is composed of
one polycarbonate layer as disk substrate, which may be a DVD; and
a glass substrate with tissue sections, which are bonded together with a mounting medium.

16. The apparatus of claim 13 wherein the modified spinning disk substrate for mounting 2D or sliced 3D tissue structured is composed of
two transparent polycarbonate layers as two separate disk substrates, which are bonded together with UV-cured adhesive;
spacers which determine the height of the spacing between the polycarbonate layers so as to form assay chambers, said spacers being substantially aligned to stabilize the rapid spinning motion
said chambers which consists of tissue sections bonded together with a mounting medium.

17. The apparatus of claim 15 wherein the mounting medium can be Fluorogel.

18. The apparatus of claim 1 wherein the 3D tissue structure can be spun with a spinning 2D field-of-view plus a sequential axial scan along the direction of light beam propagation, wherein the images can then be stacked and reconstructed in 3D, forming a volumetric tissue block structure.

19. The apparatus of claim 1 wherein the 3D tissue structure can be spun with a spinning 2D field-of-view only, wherein the images can then be stitched in 3D, forming a volumetric tissue block structure.

20. The apparatus of claim 14 wherein the imaging field-of-view of the modified spinning disk substrate can be viewed at a 2D frame rate of at least 10 Hz governed by the spinning rate which facilitates real-time video-rate dynamical monitoring at large-scale.

21. A method of preparing a substrate for the system of claim 1 for capturing specific objects comprising the steps of:
providing a transparent disk substrate that has been cleaned with 70% to 100% ethanol;
coating the disk with streptavidin;
applying a biotinylated secondary antibody coating on top of the streptavidin;
applying a coating of a primary antibody;
placing the objects to be assayed in wells on the disks;
incubating the disks for a period of time; and
rinsing the disks to reduce non-specific binding.

22. A method of preparing a substrate for the system of claim 1 for cell culturing, comprising the steps of:
providing a transparent disk substrate that has been cleaned with 70% to 100% ethanol;
sterilizing the disk ethanol with ultra-violet light;
depositing a mixture of culturing medium and cells onto the substrate; and
keeping the substrate in an incubator until the desired cell population is present on the substrate.

23. The apparatus of claim 2 wherein the the spinning illumination beams are formed by a rotating carrier carrying an illumination from a fiber, so as to avoid mechanical instability and a back-lash problem brought on by conventional strategies of back-and-forth or zig-zag-path scanning,
the spinning illumination achieved by the line-scan optical beam is directed to an integrated miniaturized optical assembly, which consists of graded-index (GRIN) lens, miniaturized relay (Mini grating) lens and an objective lens, mounted on a rotating carrier;
the said assembly is mounted in an enclosure;
the said illumination is provided from optical fiber which is attached to an outer edge of the rotating carrier by means of a rotatable joint; and
the said joint keeps the fiber from twisting while the carrier rotates.

24. The apparatus of claim 1 wherein the data recorder is an oscilloscope or a high-throughput data acquisition platform.

25. The apparatus of claim 1 wherein the high-throughput data acquisition platform is a graphic processing unit (GPU) and also a field programmable gated array (FPGA).

26. The apparatus of claim 1 wherein endogenous or intrinsic parameters retrieved from images of bioassays may be at least one of the following: optical, physical and mechanical properties of the biological specimens.

27. The apparatus of claim 26 wherein the optical property of the biological specimens may be at least one of light scattering or refractive index, the physical property of the biological specimens may be at least one of size or morphology, and the mechanical property of the biological specimens may be at least one of mass density, stiffness or deformability, traction and adhesion force.

28. The apparatus of claim 1 wherein the specimen includes standard molecular biomarkers.
